(19) United States
(12) Patent Application Publication
(10) Pub. No.: US 2006/0058695 A1
(43) Pub. Date: Mar. 16, 2006

(54) Apparatus of an improvement on the structure of chest cardiogram sensor

(76) Inventor: Tong-Pie Chen, Sanchung (TW)

Correspondence Address: BIRCH STEWART KOLASCH & BIRCH, PO BOX 747, FALLS CHURCH, VA, 22040-0747, US

(21) Appl. No.: 10/937,297

(22) Filed: Sep. 10, 2004

(51) Int. Cl. A61B 5/0402

(52) U.S. Cl. 600/509

(57) ABSTRACT

An apparatus of an improvement on the structure of chest cardiogram sensor fastened directly on the user's chest combines from shell`electric board`two fixed belts`two or multiple pieces of independent electric conductivity rubber plate, by which electric board fitted in the shell contains electricity measurement circuits and emitter circuits, whereas measurement circuits could send measured cardiogram messages to the receiver through the emitter circuits by means of cable or cableless device, and meets the requirement of monitor the user's cardiogram.

### DESCRIPTION

1. Field of the Invention

The present invention is relates to an apparatus of an improvement on chest cardiogram sensor, especially refer to a device of cardiogram sensor fasten on chest and transmit measured digital signal of heartbeat by wireless or cable to receiver.

2. Background of the Invention

Human heart is organized from muscles which is different from usual muscles and called cardiac muscle. When cardiac muscles are in active, that means heart beat and generate electric current, cardiogram sensor detects and records electricity activity in the cardiac muscles of heart and translate into curve diagram. By the diagnosis of checking the curve reflects from the cardiogram sensor, heart disease and pathogenic like arrthymia, etc. can be found out immediately. That is, from the diagnosis of checking cardiogram curve, we can judge if the heart is regular or not. Arrthymia always caused by the problem of heart conductive system, a doctor can judge from the curve shown by the cardiogram sensor to analysis and find out which part of the heart organ may have problem; In addition, the heart status may be altered by various kinds of heart diseases and shown different cardiograms on the sensor. The burden of heart workload increased by the reason of long-term high blood pressure or valvular disease will cause thicker cardiac muscles to care about the premium workload, whereas under such condition the heart electricity activity changes and also changes the curve of the heart cardiogram, therefore checking the curve of the heart cardiogram can diagnose if the cardiac muscle is thicker or not, and by the way indirectly detects some other heart diseases which increase workload of cardiac muscles. When in acute cardiac muscle infarction, cardiogram sensor will also show special apparent curves, hence heart examination with cardiogram sensor is an important therapy to the patient of cardiac muscle infarction, even in the pre-stage of cardiac muscle infarction, the chest pang or coronary hypogenesis caused by coronary shrink will also detects by cardiogram sensor. In general, cardiogram sensor provides doctor many valuable information concerning related patients.

Accordingly, the present invention provides a portable apparatus which could fasten on chest to read the body digital signal of cardiogram, and send by wireless or cable cord to its receiver. It is not only monitor the user's cardiogram from time to time, but also ready and convenient to be used outside of hospital.

### SUMMARY OF THE INVENTION

It is therefore a primary object of the present invention to provide an apparatus of improvement on the structure of chest cardiogram sensor.

It is the further object of the present invention to provide a portable and instantly measurement of cardiogram data of chest type cardiogram sensor mechanism, for convenient use and assembly.

The apparatus in accordance with the present invention primarily comprises a shell, electric board, two fixed belt, and two indecent mutual unconductivity rubber plate, and the shell is combined from bottom shell and top shell, and at the right and left edge of the bottom fixed two or multiple independent unconductivity metal plate or some other related conductive materials. Whereas, the electric board included measurement circuits and emitter circuits assemble into the shell, at both edges of circuit board contact respectively with above mentioned metal plate or some other related materials by conductive rubber or metal plate. At the bottom of fixed belt set up two or multiple independent mutually unconductive long rubber conductive plates, while the fixed belt fitted at both edges of the shell to make the outer edge of the long metal plate press under both edge of bottom shell, and electrode of measurement circuit board connect with opposite conductive rubber plates to avoid mutual conductive.

According to the description mentioned above, directly fasten the cardiogram sensor on the appropriate position of user's chest. Owing to the two sets of conductive rubber plate under the right and left ends of fixed belt connected with the electrode of measurement circuits respectively, and the conductive rubber plate further contacts with skin to get the signal data of heart electric activity, and send to measurement circuit board for calculation and translate the signal data into the digital signal, then transmit the digital signal to receiver so as to show the cardiogram. The receiver could fasten on the wrist or waist, not only portable, but also convenient.

### DETAILED DESCRIPTION OF THE PREFERRED EMBODIMENTS

Embodiments of the present invention will be described herein below by reference to the accompanying drawings and an adequate practical example:

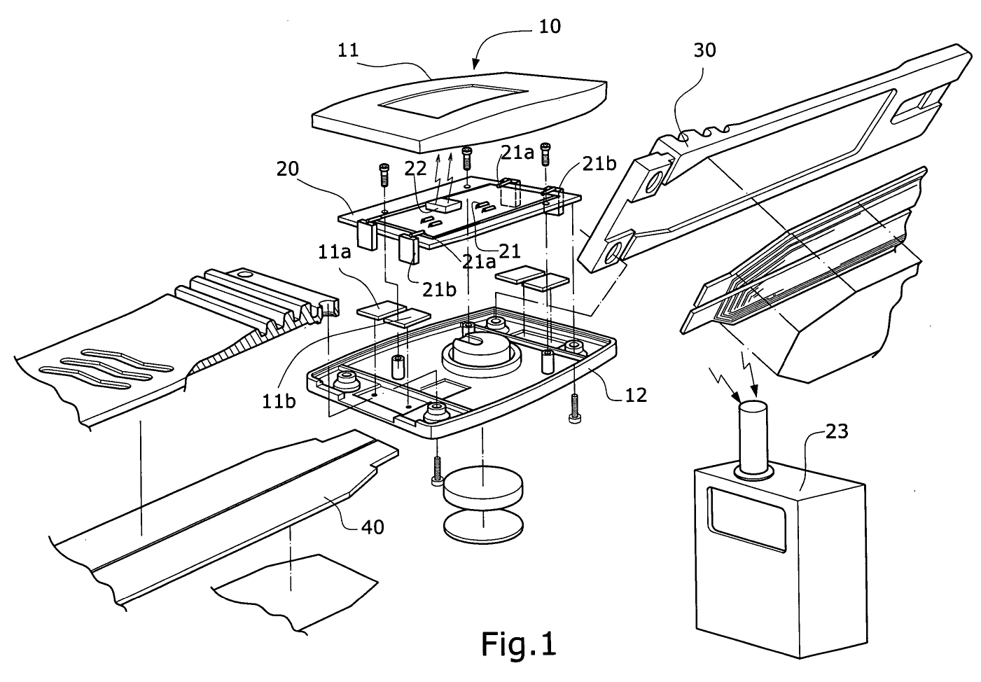

First of all, referring to FIGS. 1, 2, 3, the apparatus in accordance with the present invention primarily comprise a shell 10, circuit board 20, two fixed belt 30, and two independent mutual unconductivity rubber plate 40 at both right and left end of the fixed belt 30, among which the shell 10 combined from bottom shell 12 and top shell 11, and at the right and left edge of the bottom fixed two or multiple independent unconductivity metal plate 11a or some other related conductive materials.

Whereas, circuit board 20 includes measurement circuits 21 and emitter circuits 22, among which the two electrodes 21a at both ends of measurement circuits 21 designed to connect with conductive rubber plate or metal piece 21b, when assemble circuit board 20 into shell 10, each conductive rubber plate or metal piece 21b of electrode 21a contact with metal plate 11a; and then the emitter circuits 22 function as sending measuring data to the receiver 23 which can be fasten on wrist or waist.

Furthermore, fixed belts 30 arrange at both ends of the shell 10 and press the outer end of conductive rubber plate 40 onto the metal plate 11a at the shell 11 bottom so that conductive rubber plate can contact with circuit board 20 to form a loop circuit.

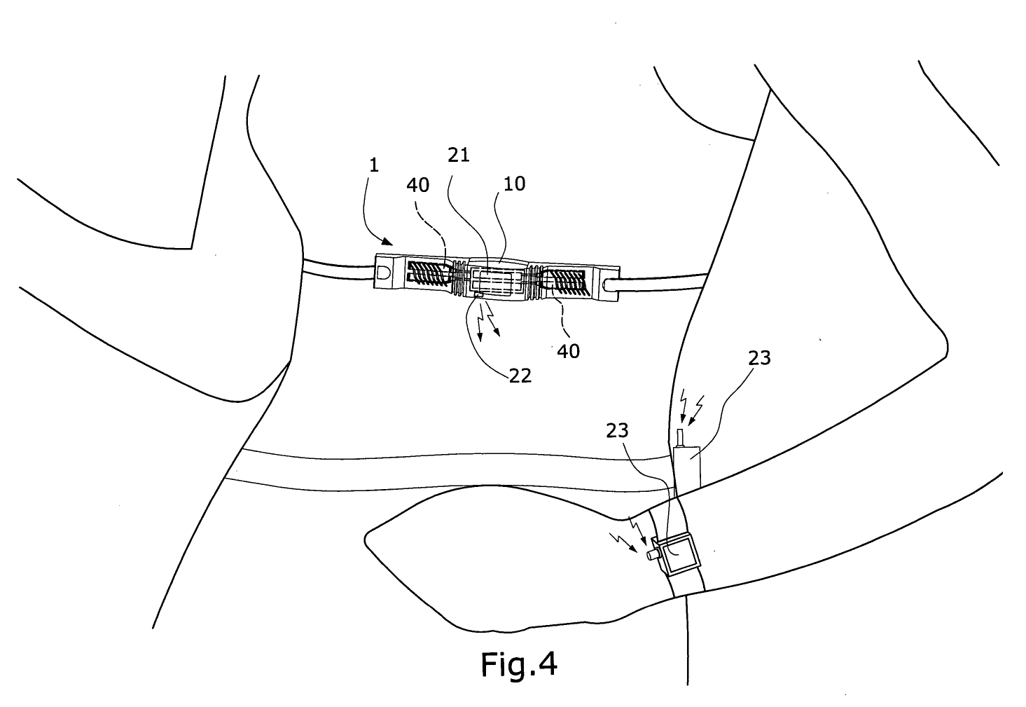

Please also refer to FIG. 4, an implementation of present invention directly fasten the cardiogram sensor on the appropriate position of user's chest. The two sets of conductive rubber plate 40 connected with measurement circuits 21 respectively, and the conductive rubber plate 40 further contacts with skin to get the signal data of heart electric activity, and circuit board 20 translate the signal data into the digital signal, then transmit the signal to receiver 23 via emitter circuits 22 of the shell 10 so as to show the cardiogram or curve.

In summary, the present invention “an apparatus of improvement on the structure of chest cardiogram sensor” fixed measurement circuits in the electric board to measure and get the calculated cardiogram data, and send to receiver which is portable for carry, assemble easily, convenient to use and receive cardiogram data immediately. Many changes and modification in the above-mentioned embodiments of the invention can, of course, be carried out without departing from the scope thereof. Accordingly, to promote the progress in science and the useful arts, the invention is disclosed and intended to be limited only by scope of the appended claim.

### BRIEF DESCRIPTION OF THE DIAGRAM

FIG. 1 is a perspective view of the present invention

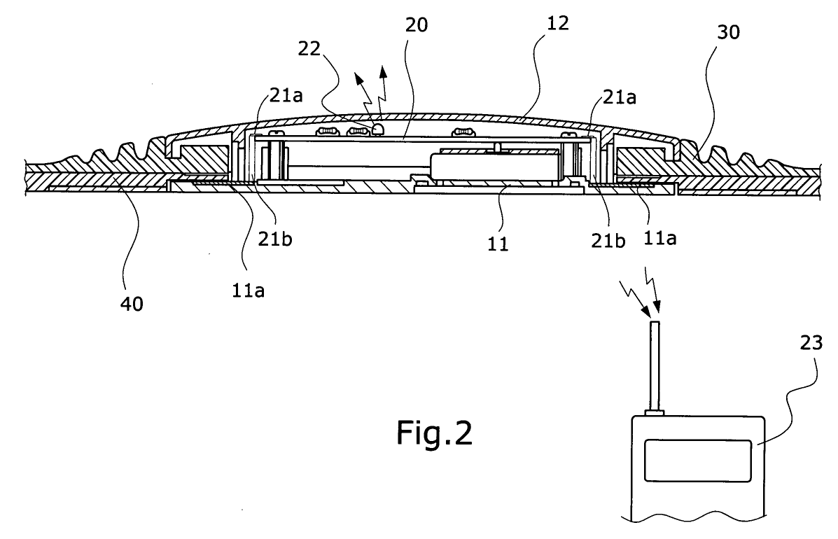

FIG. 2 is a perspective view of an assembled cardiogram sensor

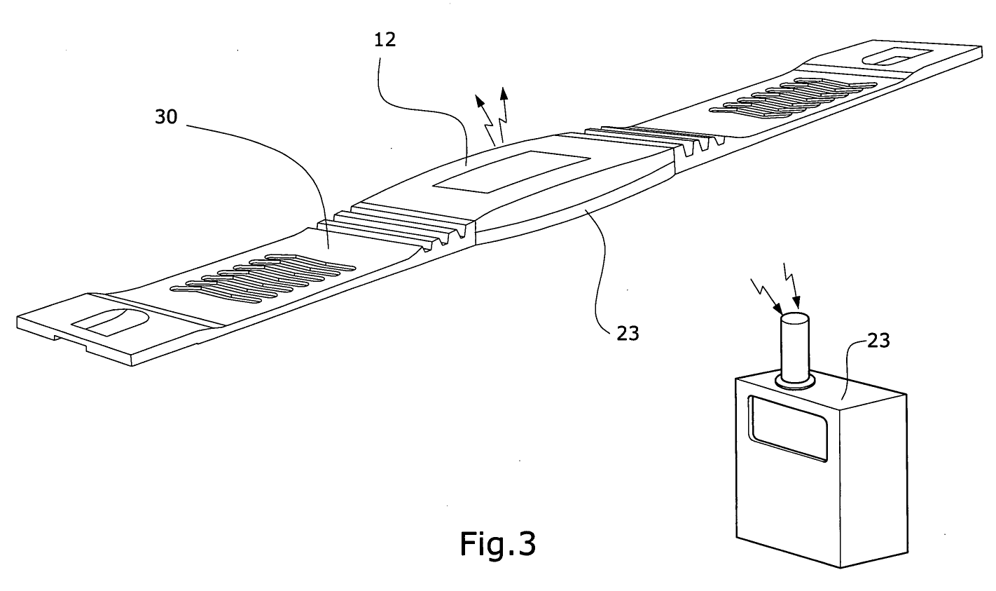

FIG. 3 is a cross sectional assembled perspective view of the present invention

FIG. 4 an implementation of the present invention
1—cardiogram sensor 10—shell
20—circuit board 30—fixed belt
40—conductive rubber plate 12—bottom shell
11—top shell 11a—metal plate
21—measurement circuits 21a—electrode
21b—conductive rubber or metal plate 22—emitter circuit
23—receiver

What is claimed is:

1. An apparatus of an improvement on the structure of chest cardiogram sensor, the apparatus in accordance with the present invention primarily comprise a shell, circuit board, two fixed belt, and two independent mutual unconductivity rubber plate, among which the shell combined from bottom shell and top shell, and at the right and left edge of the bottom fixed two or multiple independent unconductivity metal plate opposite the above mentioned rubber plate.
Whereas, circuit board includes measurement circuits and emitter circuits, among which the two electrodes at both ends of measurement circuits possessed two conductive rubber plate or metal piece, when assemble circuit board into shell, each electrode of conductive rubber plate can contact with metal plate; and then the emitter circuits can send measuring data to the receiver
Furthermore, fixed belts arrange at both ends of the shell and press the outer end of conductive rubber plate onto the metal plate at the shell bottom, so that conductive rubber plate can contact with circuit board to form a loop circuit.

2. An apparatus of an improvement on the structure of chest cardiogram sensor according to claim 1, wherein the conductive rubber plates of electrode at both ends of measurement circuits could be changed into metal plate.

3. An apparatus of an improvement on the structure of chest cardiogram sensor according to claim 1, wherein metal plate can be changed into another materials which is electric conductivity.
